(19) United States
(12) Patent Application Publication
(10) Pub. No.: US 2026/0301857 A1
(43) Pub. Date: Oct. 1, 2026

(54) Computational Method to Identify Protein Hotspots and Allosteric Sites

(71) Applicants: Paul Campitelli, Tempe, AZ (US); Tushar Modi, Woburn, MA (US); Sefika Banu Ozkan, Phoenix, AZ (US)

(72) Inventors: Paul Campitelli, Tempe, AZ (US); Tushar Modi, Woburn, MA (US); Sefika Banu Ozkan, Phoenix, AZ (US)

(73) Assignee: Arizona Board of Regents on Behalf of Arizona State University, Scottsdale, AZ (US)

(21) Appl. No.: 19/631,826

(22) Filed: Mar. 27, 2026

Related U.S. Application Data
(60) Provisional application No. 63/779,777, filed on Mar. 28, 2025.

(51) Int. Cl. G16B 15/20; G16B 5/30; G16B 15/30; G16B 20/50; G16B 40/30

(52) U.S. Cl. CPC G16B 15/20; G16B 5/30; G16B 15/30; G16B 20/50; G16B 40/30

(57) ABSTRACT

Disclosed herein are systems and methods for characterizing a protein, the systems and methods including: accessing simulated protein structure data with a computer system, in which the simulated protein structure data indicate a structure of the protein; clustering, using the computer system, the simulated protein structure data to produce a plurality of clustered structures and determining an equilibrium of each clustered structure in the plurality of clustered structures; calculating, using the computer system, time-dependent responses of each clustered structure in the plurality of clustered structures; identifying, using the computer system, at least one residue of interest based on the time-dependent responses of each clustered structure in the plurality of clustered structures; and characterizing, using the computer system, the at least one residue of interest and the protein.

### CROSS-REFERENCE TO RELATED APPLICATIONS

This application claims the benefit of and priority to U.S. Application No. 63/779,777, filed Mar. 28, 2025, the entirety of which is incorporated herein by reference.

### STATEMENT REGARDING FEDERALLY SPONSORED RESEARCH OR DEVELOPMENT

This invention was made with government support under GM147635 awarded by the National Institutes of Health. The government has certain rights in the invention.

### BACKGROUND

Protein allostery, where changes at one site affect function at distant sites, plays a fundamental role in biological processes and drug development. Understanding these mechanisms is advantageous for therapeutic intervention. Current methods primarily focus on equilibrium dynamics and network analysis to map allosteric interactions. However, these approaches are limited by their reliance on steady-state analysis and subsequent time-dependence, missing relevant temporal aspects of allosteric communication. Thus, there exists a need for new approaches combining molecular dynamics trajectories with time-dependent linear response theory, enabling the analysis of dynamic responses to perturbations over time.

### SUMMARY

Disclosed herein are apparatus, systems, and methods for characterizing a protein. The systems and methods may include: accessing simulated protein structure data with a computer system, in which the simulated protein structure data indicate a structure of the protein; using the computer system to: cluster the simulated protein structure data to produce a plurality of clustered structures and determining an equilibrium of each clustered structure in the plurality of clustered structures; calculate time-dependent responses of each clustered structure in the plurality of clustered structures; identify at least one residue of interest based on the time-dependent responses of each clustered structure in the plurality of clustered structures; and characterize the at least one residue of interest and the protein.

In some embodiments, accessing the simulated protein structure data comprises simulating the structure of the protein with the computer system using isobaric canonical ensemble MD simulations.

In some embodiments, calculating the time-dependent responses of each clustered structure in the plurality of clustered structures includes: extracting cross velocity-positional covariance matrices corresponding to each clustered structure in the plurality of clustered structures; and applying perturbative forces to the cross velocity-positional covariance matrices corresponding to each structure in the plurality of clustered structures to calculate the time-dependent responses.

In some embodiments, identifying the at least one residue of interest comprises using frequency domain analysis. In some embodiments, identifying and characterizing the at least one residue of interest comprises identifying the at least one residue as allosteric or non-allosteric.

In some embodiments, the method is used to predict a mutational impact on the protein. Predicting the mutational impact on the protein may include: selectively mutating the protein using single amino acid substitutions; repeating the steps of the method on the selectively mutated protein; and comparing the characterization of the selectively mutated protein and the protein from step a.

In some embodiments, the method is used to investigate antibiotic resistance. In some embodiments, the method may include generating a report including the characterization of the at least one residue and the protein.

### BRIEF DESCRIPTION OF THE DRAWINGS

The disclosure will hereafter be described with reference to the accompanying drawings, wherein like numerals denote like elements.

FIG. 1 shows a diagram showing construction of time-dependent response profiles using 3-D structure TEM-1 (PDB ID 1×PB (24)) showing time-dependent perturbative force applied to a position j (here, active site S70) and subsequent time-resolved perturbation response at position i (here, residue D35). Average response profile per time step is shown in dark blue and the variance per time step is shown in lighter blue. Representative plots for time versus unit pulse force applied to position j and response profile of position i show an example case, using pulse intervals of 5 ns.

FIG. 2 shows a schematic diagram outlining the process of constructing time-resolved perturbation response profiles after the first equilibrium simulations.

FIGS. 3A-3C show (FIG. 3A) Response magnitude over time and (FIG. 3B) Fourier-transformation of the response magnitude for four positions: two non-allosteric positions (L55 and A150) and two allosteric positions (V44 and S203) as a result of pulse perturbations applied to active site S70 at 1 ns intervals. (FIG. 3C) Locations of these positions are shown as a carbon spheres on the TEM-1 structure (PDB ID: 1BTL (33)). We see that the direct perturbation and time-dependent response approach is able to separate out the allosteric from non-allosteric residues by their response profiles (FIG. 3A) but through the transformation into frequency space (FIG. 3B), inspection of the lowest frequency regimes shows a clean separation between this sample set of allosteric and non-allosteric residues. For each position, the variance in response per time step (FIG. 3A) is shown as semitransparent and the darker colors represent the average response per time step.

FIGS. 4A-4F show PCA of a larger set of allosteric (V44, S203, A232, A249, V262, and 286; red) and non-allosteric (K55, A150, P226, and K256; blue) positions from the first two PCs of Fourier-transformed response profiles of perturbations at active sites S70 (FIG. 4A, FIG. 4D), S130 (FIG. 4B, FIG. 4E), and E166 (FIG. 4C, FIG. 4F). Using hyperplane distance separating the two groups as a metric, we rank the best [top (FIG. 4A-FIG. 4C)] pulse intervals per position and worst [bottom (FIG. 4D-FIG. 4F)] pulse intervals per position. Solid black line represents the hyperplane, and the dashed lines are the hyperplane margins, calculated using SVM. While, overall, these first two PCs do an adequate job separating out this set of allosteric from non-allosteric residues, different pulse intervals applied to different functionally important sites tend to provide better or worse separation.

FIG. 5 shows a First PC (e.g., PC1) obtained from PCA of the Fourier-transformed responses of each residue position to pulse interval perturbations to the specific three active sites S70 (20.5 ns), S130 (1.0 ns), and E166 (14.5 ns) in the TEM-1 protein. Each dot represents one residue position and is colored by their average change in AMP fitness values measured at 625 μg/mL. Projections onto the X-Y plane are shown as shadows for additional visual clarity.

FIGS. 6A-6D show (FIG. 6A) Accuracy and (FIG. 6B) TPR as a function of the total number of features used as input for logistic regression analysis of AMP and CTX fitness (see Section Methods for descriptor of class designation). It should be noted that a higher accuracy of CTX class identification is, to some extent, related to the relative number of true positives as compared to AMP (35 vs 159), as shown in (FIG. 6B). (FIG. 6C, FIG. 6D) Individual plots of probability vs class for (FIG. 6C) AMP at 95% accuracy and (FIG. 6D) CTX at 100% accuracy using 20 and 19 total features, respectively, along with the feature breakdown by perturbed position and pulse interval in nanoseconds.

FIG. 7 shows Probabilities extracted from the AMP logistic regression model compared to the experimental average ΔAMP fitness values at 625 μg/mL concentration. These quantities are reasonably well-correlated, with a Pearson R value=0.79.

FIGS. 8A-8C show a complement FIG. 2. (FIG. 8A) Response magnitude over time and (FIG. 8B) Fourier-transformation of the response magnitude for four positions: Two non-allosteric positions (L55, A150) and two allosteric positions (V44, S203) as a result of pulse perturbations applied to active site S70 at 5 ns intervals. The locations of these positions are shown as alpha carbon spheres on the 3D TEM-1 structure (PDB ID: 1BTL1) in (FIG. 8C). We see that the direct perturbation and time-dependent response approach is able to separate out the allosteric from non-allosteric residues by their response profiles (FIG. 8A) but through the transformation into frequency space (FIG. 8B), inspection of the lowest frequency regimes shows a clean separation between this sample set of allosteric and non-allosteric residues. For each position the variance in response per timestep (FIG. 8A) is shown as semi-transparent and the darker colors represent the average response per timestep. It should be noted that, as expected, the response magnitude of all positions under 5 ns pulse interval conditions is lower than Ins pulse interval conditions, as less perturbative force is applied to the system over the 100 ns window.

FIG. 9 shows a complement to main text FIG. 5. First principal component (e.g., PC1) for every position in the TEM-1 system obtained from PCA of the Fourier-transformed responses to pulse interval perturbations to the specific active sites K73 (0.75 ns), S130 (1.0 ns) and K234 (14.25 ns). Each dot represents one position and is colored by their average change in AMP fitness values measured at 625 μg/ml. Projections onto the X-Y plane are shown as shadows for additional visual clarity. This general trend between ampicillin fitness and principal components exists for any three active sites perturbed.

FIG. 10 shows a complement to FIGS. 3A-3C, FIG. 5, and FIG. 9. Allosteric residues as used in FIGS. 3A-3C are shown as red dots, all others are blue. (Left) Correlation analysis and Pearson R between first principal component (e.g., PC1) values (used in FIGS. 3A-3C, FIG. 5, and FIG. 9 analyses) and distance to the corresponding perturbed active site. While a general trend between active site distance and PC value exists, overall these correlations are rather weak. (Right) Correlation analysis and Pearson R between PC1 values and the change in Ampicillin fitness. Each PC alone correlated reasonably well with Ampicillin fitness but fails to capture the substitution behavior as strongly as exhibited in FIG. 5.

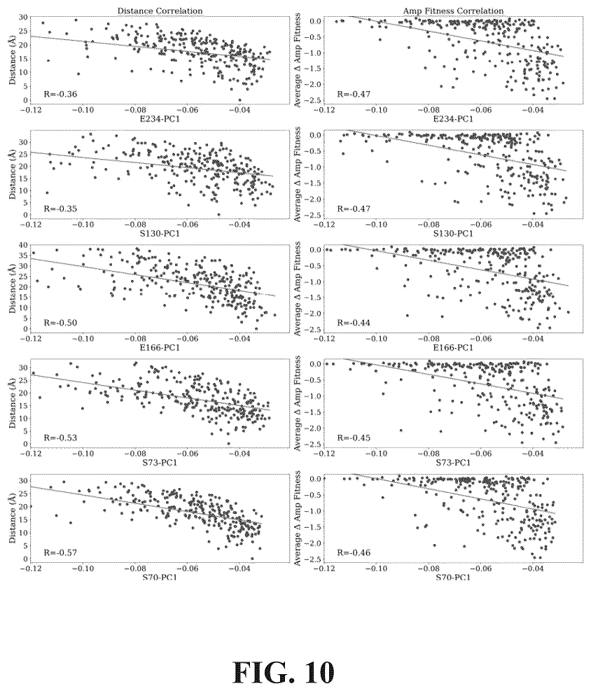

FIG. 11 shows an example process of characterizing a protein in accordance with some embodiments of the disclosure.

FIG. 12 shows an example system in accordance with some embodiments of the disclosure.

FIG. 13 is a list of features used in each logistic regression classification model.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

The present disclosure provides a comprehensive computational method for understanding protein allostery and regulation through a novel combination of molecular dynamics and time-dependent linear response theory. By examining the time evolution of residue fluctuation responses to force perturbations at functional sites, the disclosed methods and systems offer essential insights into allosteric communication within the protein interaction network through protein dynamics. The disclosed methods and systems further reveal previously undetectable aspects of allosteric communication by examining both temporal and frequency domains of protein responses. The application of the disclosed methods on different proteins can be crucial in identifying regulatory sites in proteins, predicting mutational effects, and developing effective therapeutic strategies targeting allosteric regulation in various disease-relevant proteins.

Exemplary systems and methods described herein employ a cutting-edge combination of computational methods to unveil the molecular mechanisms underlying protein allostery. The process may involve the following elements:

Time-Dependent Protein Dynamics Analysis: The method applies pulse perturbations at various intervals to active sites and analyzes the dynamic response of each residue over time. This reveals the temporal nature of allosteric communication through response profiles that distinguish allosteric from non-allosteric residues.

Frequency Domain Analysis: Fourier series analysis transforms time-domain data into the frequency domain, providing additional insights into the dynamics of allosteric interactions. This transformation enables the separation of significant allosteric influences from non-allosteric positions through analysis of principal components.

Machine Learning Integration: Classification models built on time-dependent perturbation responses can identify regulatory positions with high accuracy. These models can predict the functional impact of mutations and identify key regulatory sites without extensive experimental testing.

Antibiotic Resistance Analysis: The method connects time-resolved protein dynamics to antibiotic resistance mechanisms by analyzing how perturbation responses correlate with resistance phenotypes. This enables the identification of positions that regulate antibiotic resistance and provides insights into the molecular basis of resistance, as demonstrated through successful application to TEM-1 β-lactamase and its resistance to various antibiotics.

The integration of time-dependent linear response theory with molecular dynamics to provide a comprehensive understanding of protein allostery has not been previously investigated. By employing unique computational approaches that analyze both temporal and frequency domains, we can capture the dynamic nature of allosteric communication and reveal previously hidden aspects of protein regulation. This method offers mechanistic insights into how proteins transmit information across long distances and how mutations can affect these communication networks. This knowledge can be leveraged to inform the development of more effective therapeutic strategies targeting protein regulation in various diseases as well finding novel drugs targeting allosteric (hidden pockets controlling active sites).

Some advantages of the disclosed methods include but are not limited to the following. In some embodiments, the systems and methods described herein may provide temporal resolution of allosteric communication, revealing aspects of protein regulation that are invisible to traditional stead-state approaches. In some embodiments, the analysis of frequency domains through Fourier transformations with the systems and methods described herein allows for robust interpretation of complex protein dynamics. In some embodiments, the systems and methods described herein may predict the functional impact of mutations without requiring extensive experimental validation. In some embodiments, the systems and methods described herein may be used to identify novel drugs targeting hidden allosteric pockets as an alternative approach to drug repurposing. In some embodiments, the systems and methods described herein are protein-agnostic and may be applied to any protein system with an available structure.

In some embodiments, the systems and methods described herein may be applied in drug discovery, protein engineering, and therapeutic developments. In some embodiments, the systems and methods described herein may identify a novel allosteric drug target in a protein and/or protein system (e.g., an oncogenic protein and/or protein system). In some embodiments, the identification of the novel allosteric drug target may lead to a more effective therapeutic. In some embodiments, the systems and methods provide the ability to understand temporal aspects of protein regulation. In some embodiments, understanding temporal aspects of protein regulation may lead to a more effective therapeutic and/or therapeutic strategy. In some embodiments, the protein and/or protein system is associated with a disease. In some embodiments, the disease is cancer. In some embodiments, the disease is a neurodegenerative disease (e.g., Alzheimer's, Parkinson's, amyotrophic lateral sclerosis, Huntington's, or multiple sclerosis).

In some embodiments, the systems and methods described herein may guide the rational design of proteins. In some embodiments, the rationally designed proteins may have an enhanced and/or modified function relative to the native protein. In some embodiments, the systems and methods described herein predict the impact of a mutation on protein regulation. In some embodiments, the predicted mutational impact enhances and/or modifies the function of the rationally designed protein relative to the native protein. In some embodiments, the use of the systems and methods described herein to rationally design proteins may be used in an application in biotechnology, biocatalysis, and/or developments of a therapeutic (e.g., a protein-based therapeutic).

In some embodiments, the systems and methods described herein may be applied to understand a drug resistance mechanism. In some embodiments, the systems and methods described herein may be applied to design a better therapeutic antibody. In some embodiments, the systems and methods described herein may be applied to develop a novel approach to modulate protein function.

The systems and methods described herein are protein-agnostic. The protein-agnostic nature of the systems and methods described herein makes it particularly valuable for addressing a wide range of therapeutic targets across different disease areas.

In some embodiments, systems and methods for characterizing a protein are described herein. The method may include using a computer system to: access simulated protein structure data, in which the simulated protein structure data indicate a structure of the protein; cluster the simulated protein structure data to produce a plurality of clustered structures and determine an equilibrium of each clustered structure in the plurality of clustered structures; calculate time-dependent responses of each clustered structure in the plurality of clustered structures; identify at least one residue of interest based on the time-dependent responses of each clustered structure in the plurality of clustered structures; and characterize the at least one residue of interest and the protein.

The simulated protein structure data may be generated using MD simulations. For instance, the MD simulations may be all-atom, isobaric canonical ensemble MD simulations.

In some embodiments, clustering the simulated protein structure data can be completed using k-means clustering, density-based clustering, centroid-based clustering, or any other suitable clustering method.

In some embodiments, calculating the time-dependent response of each clustered structure in the plurality of clustered structures includes extracting cross velocity-positional covariance matrices corresponding to each cluster, and applying perturbative forces to the cross velocity-positional covariance matrices. In some embodiments, the perturbative forces are applied at a specified pulse interval. In some embodiments, the perturbative forces are applied at more than one specified pulse interval. A “time-dependent response” may also be referred to as a time-dependent response profile.

In some embodiments, identifying a residue of interest includes using frequency domain analysis. This may include using a Fourier transformation of the time-dependent response profiles. The corresponding Fourier transform can provide additional information not accessible by simply viewing a time-dependent response. For instance, different decay rates in the Fourier transform can provide information on the rates of kinetic interactions between residues. Additionally, using a Fourier transform can more easily distinguish allosteric and non-allosteric residues, as well as distinguishing interactions between allosteric and catalytic residues. In some embodiments, dimension reduction methods such as PCA may be used to further analyze the Fourier transform.

In some embodiments, the methods and systems described herein may be used to predict mutational impact on the protein. In these methods, a protein may be mutated using single amino-acid substitutions, or multiple amino-acid substitutions. The protein may then be simulated and analyzed as described above. In some embodiments, the amino-acid substitutions occur at sites that have been identified as sites of interest. In some embodiments, the mutated protein and non-mutated protein may be compared.

In some embodiments, the methods and systems described herein may be used to investigate antibiotic resistance. For instance, the methods may be used to identify residues that are important for binding to antibiotics, and used to predict effects of mutating the identified residues on binding to antibiotics. The methods may be used in a variety of protein therapeutics, including but not limited to, antibodies, antibody-drug conjugates, recombinant proteins, fusion proteins, insulin, growth hormone, somatotropin, mecasermin, Factor VIII, Factor IX, antithrombin III, β-Gluco-cerebrosidas, alglucosidase-α, laronidase, lactase, human albumin, or pancreatic enzymes.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, or example, by a mutated residue. A protein, peptide, or polypeptide may also be a single molecule or may be a multi-molecular complex. A protein, peptide, or polypeptide may also be a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain.

A “site” or “residue” may refer to an amino acid at a specific position in the protein. The terms may be used to refer to the position, or the specific amino acid at said position. The terms site and residue are used interchangeably throughout the specification.

### EXAMPLES

### Example 1: Dissecting Allosteric Mutations for Antibiotic Resistance by Time-Dependent Linear Response Theory

We report a new approach that combines molecular dynamics trajectories with time-dependent linear response theory to compute the time evolution of residue fluctuation responses to force perturbations exerted at functional sites. Applying this new approach to TEM-1 β-lactamase, we observe that the time-resolved response profiles of allosteric sites to perturbations of TEM-1 active sites are distinct from those of non-allosteric residues. Using Fourier transformations, we convert the time domain response profiles to the frequency domain and demonstrate that the frequency space representation of the perturbation response can capture the mutational behavior of each site when applied to deep sequencing mutational data. Furthermore, we show that classification models built on perturbation responses can accurately identify distal positions that regulate antibiotic resistance. These findings provide insights into the contributions of specific residues to resistance-encoded in time-resolved perturbation response behavior and highlight the importance of this new approach in identifying allosteric mutations, opening avenues for the potential characterization of additional allosteric positions without extensive computational simulations.

Understanding the intricate mechanisms underlying protein function and regulation is essential for the elucidation of cellular processes. Allostery, the phenomenon where a perturbation at one site on a protein induces a functional response at a distant site, plays a crucial role in many biological processes. (1-10) Identification and characterization of allosteric sites are of paramount importance, as they offer potential targets for therapeutic intervention. (10-15)

Over the years, numerous studies have focused on developing methods to identify and unravel the modulating effects of allosteric sites. Current methods often rely on network analysis, employing dynamic or static models to infer allosteric interactions and create edges between residues to map allosteric networks. These approaches, such as Ohm (16) and time-independent component analysis, (17-19) have provided valuable insights into the allosteric landscape of proteins. Allosteric interactions have also been understood through coupling strength and asymmetry in coupling strength, analyzed through the dynamic coupling index (DCI) and DCI asymmetry in a variety of protein systems. (4, 11, 20, 21) By analyzing equilibrium dynamics, researchers have successfully mapped out allosteric networks and identified key residues involved in long-range signaling. However, these methods primarily address the equilibrium state of proteins, and there is growing recognition of the need to explore the time and frequency regimes to further differentiate allosteric behavior.

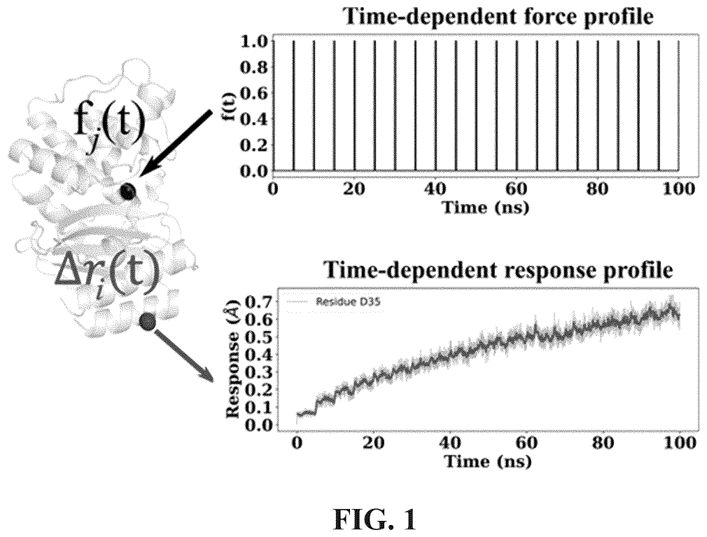

In this study, we propose a novel approach that goes beyond the limitations imposed due to equilibrium dynamics by employing the time-dependent linear response theory (22,23) to efficiently evaluate long distance coupling and provide mechanistic insights on allosteric regulations. By considering the dynamic response of a protein to perturbations over time, we gain a deeper understanding of the time-dependent nature of allosteric communication. Our method involves applying pulse perturbations at various intervals to active sites and analyzing the ensuing dynamic response of each residue (FIG. 1). Comparison of the time of evolution of the response profiles to periodic pulse perturbations exerted at active sites allows us to separate out distinct test cases of allosteric versus non-allosteric sites, enabling us to observe the time dependence of allostery for the first time.

To evaluate each position's contribution toward long distance regulation, we employ Fourier series analysis, a powerful tool in signal processing. By transforming the time-domain data into the frequency domain, we gain additional insights into the dynamics of allosteric interactions. Specifically, we can separate out larger allosteric influences from non-allosteric positions by analyzing the principal components (PCs) of these Fourier series. This approach not only provides a more detailed picture of the underlying dynamics but also enables us to identify key frequency components associated with allosteric communication.

To demonstrate the effectiveness and broad applicability of our approach, we further expand its application to a comprehensive deep mutational scanning (DMS) dataset associated with ampicillin (AMP) and cefotaxime (CTX) resistance in TEM-1 β-lactamase. (25) TEM-1 is a well-studied system; the mutational landscape has been explored comprehensibly, with various computational and experimental approaches. (26-31) Remarkably, even when applied solely to the dynamics analysis of wild-type (WT) TEM-1 β-lactamase, our method successfully captures the fundamental mutational behaviors of each position. By analyzing the time evolution of the fluctuation response of each position to pulse perturbations to active sites, we can extract meaningful insights into the allosteric effects of specific mutations. This proof of concept showcases the versatility and robustness of our approach in capturing the essential mutational behaviors associated with antibiotic resistance. It should be noted that all of the work presented here focuses on the apo (unliganded) TEM-1 system. We expect there to be noticeable response profile changes between bound and unbound forms of not only TEM-1 but also any given protein system.

In summary, our paper presents a unique and innovative approach for investigating allosteric interactions by leveraging time-dependent linear response theory. By considering the time and frequency regimes of equilibrium dynamics, we shed new light on the dynamic nature of allostery. Through experimental validation on the TEM-1 β-lactamase and an expanded mutational dataset, we demonstrate the efficacy of our method in unraveling the complex interplay between allosteric and non-allosteric sites. In particular, this new approach allows us to measure the specific time-scales/frequencies that each active site communicates with distal positions in the protein chain to regulate enzymatic function. By offering new insights into allosteric mechanisms, our findings contribute to a deeper understanding of protein regulation and open up exciting possibilities for future therapeutic interventions.

### Methods

### Time-Dependent Linear Response Theory

We here show the pertinent, related equations for the calculations of time-dependent responses of residues due to periodic force perturbations over time in the protein in 1-D; the formal derivation in 1-D is given as an example in the Supporting Information, where the generalization to the 3-D case follows exactly when taking appropriate vector products.

Under equilibrium conditions, if a force f(t) is applied, the time average response at position i at time t can be calculated as

$$〈 Δ {r}_{i} ( t ) 〉 = 〈 Δ {r}_{i} ( t ) \vert ω ( t ) 〉 . \qquad Eq 1$$

where ω(t) is the perturbed phase space with the response Δω(t)

$$ω ( t ) = {ω}_{0} + Δ ω ( t ) . \qquad Eq . 2$$

Considering H_{f}, the Hamiltonian of the perturbed protein with m particles where each particle j has an external force fⱼ(t) exerted on them

$${H}_{f} = {H}_{0} - {\sum }_{j - 1}^{m} Δ {r}_{j} {f}_{j} ( t ) . \qquad Eq . 3$$

Along with the covariance between the velocity of positions j at time 0 with the displacement of position i (calculated under the conditions of equilibrium), one can calculate the average response of a position i to perturbations at several other positions as

$$〈 Δ {r}_{j} ( t ) 〉 = \frac{1}{{k}_{B} T} {\int }_{0}^{t} {dt}^{′} {\sum }_{j = 1}^{m} {〈 Δ {\dot{r}}_{j} ( 0 ) Δ {r}_{j} ( {t}^{′} ) 〉}_{0} {f}_{j} ( t − {t}^{′} ) . \qquad Eq . 4$$

In order to calculate the response, we can use the trajectory of a protein obtained from molecular dynamic simulations. For the work presented here, 50 unit force perturbations of randomized direction were applied at a given pulse interval to a single residue j at a time and the average responses over these 50 perturbations were calculated. In cases for only one single perturbed residue j, we have our fluctuation response of residue i as

$$〈 Δ {r}_{j} ( t ) 〉 = \frac{1}{{k}_{B} T} {\int }_{0}^{t} {dt}^{′} {〈 Δ {\dot{r}}_{j} ( 0 ) Δ {r}_{j} ( {t}^{′} ) 〉}_{0} {f}_{j} ( t − {t}^{′} ) . \qquad Eq . 5$$

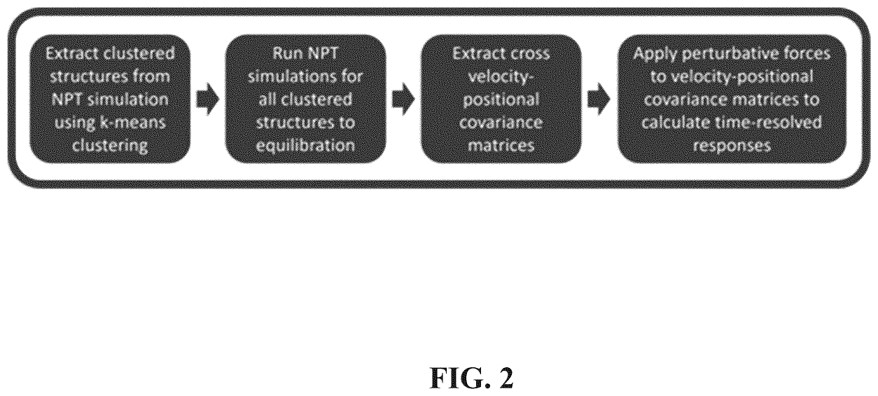

To obtain time-dependent response profiles of the positions to the periodic force perturbations exerted on the functional sites, we first run long, well-sampled all-atom MD simulations and cluster the protein conformations sampled. Using these structures, we then perform NPT ensemble MD simulations to extract cross velocity position covariance matrices (FIG. 2). Further information regarding the MD setup and protocol can be found in the section below.

### Molecular Dynamics Setup

Molecular dynamics simulations were conducted to obtain the equilibrium dynamics of TEM-1 β-lactamase using AMBER20. (32) The protein system was prepared by using the crystal structure of TEM-1 β-lactamase (PDB ID: 1BTL (33)). The simulated system is composed of a cubical box with a padding of 14A from the protein edges and explicit water molecules using the TIP3P model. (34) The solvated system was further neutralized by adding sodium (Na+) and chloride (Cl−) ions to ensure overall charge neutrality.

The prepared system underwent multiple stages of simulation, including energy minimization, equilibration, and production runs. Energy minimization was performed to relieve steric clashes and optimize the initial system energy using the steepest descent algorithm. Position restraints were applied during energy minimization to constrain the solute atoms (protein and solvent) while allowing the solvent to relax. The minimization process continued until a convergence threshold was reached. For the second unconstrained energy minimization step, the solvated system underwent the steepest descent energy minimization. A total of 100,000 cycles were performed. The SHAKE algorithm (35) was utilized to enforce constraints on the bond lengths involving hydrogen atoms.

Subsequent to the energy minimization phase, a gradual temperature increase was implemented to facilitate a heat-up procedure, transitioning the system from 0 to 300 K. The simulation encompassed a total of 50,000 steps, with a time step of 2 fs. The SHAKE algorithm (35) was employed once again to uphold constraints on hydrogen atom bond lengths. Finally, a production run was conducted for a total simulation time of 2 μs using a time step of 2 fs. Long-range electrostatic interactions were calculated using the particle mesh Ewald method. (36) Direct-sum, nonbonded interactions were cut-off at distances of 12.0 Å or greater. A constant number of particles, isothermal, and isobaric canonical ensembles (NPT) were used for the production run. The temperature was set to 300 K, and the pressure was regulated as 1 bar. To sustain the desired temperature, a Langevin thermostat was utilized with a collision frequency of 1.0 ps⁻¹. The pressure was regulated by utilizing the Berendsen barostat algorithm, maintaining a constant system volume.

From these equilibrium simulations, k-means clustering was performed on conformational diversity, from which 2 structural clusters were produced with a rejection based on a 0.8 Å cutoff from the cluster centers.

Using the cluster centroids as starting points and the above simulation protocol, a further independent NPT ensemble equilibrium production run was employed to compute the cross velocity-positional covariance matrices for each clustered structure. Thus, there are two total additional NPT simulations, one for each structural cluster. This ensures that the covariance matrices represent the contribution from the diversity of the conformational ensemble of the protein. FIG. 2 contains a schematic overview of the process to construct time-dependent perturbation response profiles starting from an initial equilibrated simulation.

Perturbations to Active Site S70 Generate Time-Resolved Response Profiles that can Distinguish Allosteric from Non-Allosteric Residues

We first wanted to determine if it would be possible to distinguish, as proof of concept, allosteric from non-allosteric positions within TEM-1 using time-resolved perturbation response profiles of positions upon active site perturbations. Previous work using time-independent dynamic analysis techniques such as the dynamic flexibility index and the DCI have been implemented on a wide range of protein systems with success in identifying and predicting allosteric positions. (1, 4, 7, 37-39) Particularly, we have seen instances in which the coupling strength between important active site positions and putative allosteric positions has differed from non-allosteric positions. DCI utilizes a perturbation response technique in which the coupling strength between any i-j pair of residues within a protein is measured by a normalized fluctuation response profile of j when position i is perturbed. As such, with the implementation of this time-dependent linear response technique, we expected to also see such distinguishing behavior upon perturbations of the TEM-1 active site positions.

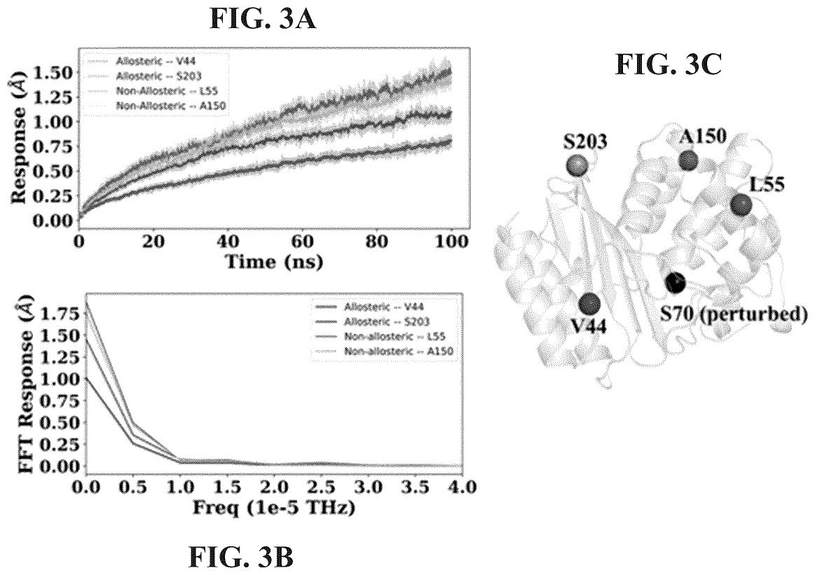

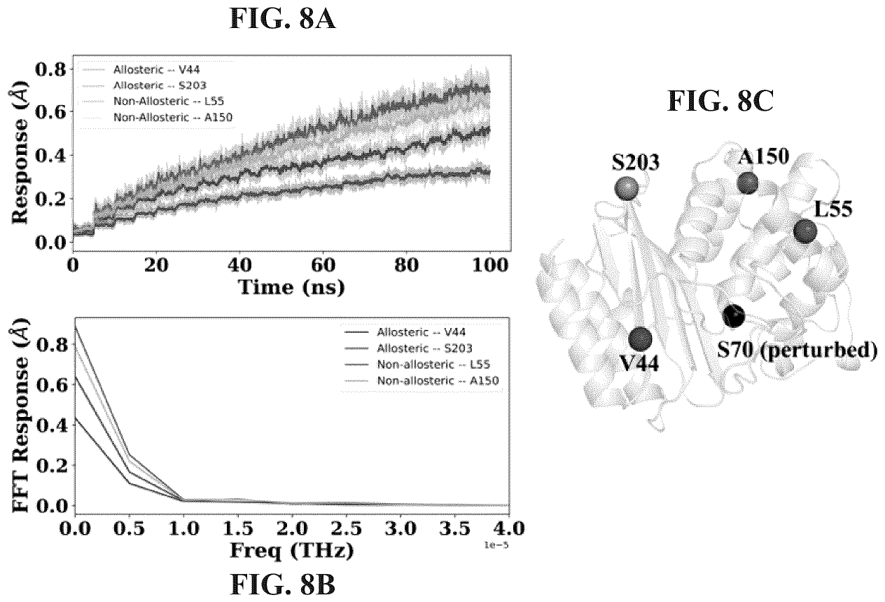

Here, we compute the position-specific fluctuation response profiles of all residues by applying force pulses to active site S70 over 100 ns cross velocity-position covariance matrices, using two pulse intervals of 1 and 5 ns. We then analyze the response profiles of four experimentally characterized allosteric sites V44 and S203 versus non-allosteric sites L55 and A150. (40) Note that while our analysis is performed on a WT TEM-1, the experimentation for the allosteric sites mentioned here was performed on the M182T TEM-1 variant background. FIG. 3A shows the response magnitudes of these positions over this 100 ns period under conditions of 1 ns pulse intervals (see FIGS. 8A-8C for 5 ns pulse intervals). There is a clear distinction of time-dependent response profiles between allosteric and non-allosteric sites; interestingly, allosteric residues converged to different response magnitudes more quickly than non-allosteric residues.

The faster convergence of allosteric residues to a given magnitude compared to non-allosteric ones can be attributed to their critical role in signal transmission within proteins. Allosteric residues are often integral components of communication pathways, and their ability to respond quickly to perturbations is essential for efficient allosteric regulation. (41) This rapid response may allow allosteric sites to act as time-dependent “hinges”, crucial for the propagation of mechanical forces, akin to joints in a skeleton.

Further, allosteric residues may have been evolutionarily optimized to respond swiftly to changes, vital for their function in signal transmission. Studies have shown that allosteric and catalytic residues often coevolve to optimize their functional relationship, enhancing the efficiency of communication and regulatory mechanisms within the protein. (42)

In addition, we extend and quantify the time-resolved responses further by applying a Fourier transform to frequency space, leveraging the time series nature of our time-dependent perturbation technique. This approach provides a robust analysis of the frequency components of the protein's response, enabling us to identify the characteristic frequencies or modes of motion distinguishing allosteric from non-allosteric residue positions. In FIG. 3B, we present the Fourier transformation of the response profiles in FIG. 3A within the 0 to 4.0×10⁻⁵ THz regime. A clear separation in the frequency domain emerges between allosteric and non-allosteric residues. Notably, a faster decay in the Fourier transform implies more rapid interaction kinetics between the allosteric and catalytic residues, a signature of dynamic allostery observed in other allosteric systems as well. (43)

Fourier Transforms Provide a Robust Way of Interpreting these Time Series Response Obtained by Periodic Perturbation of the Active Site Positions

Within the first, lowest frequency ranges, the allosteric from non-allosteric positions are able to be quite immediately distinguished from one another based on their response profiles to periodic force perturbation to catalytic site S70. The clear separation indicates that some of the important information encoding positional behavior within a protein can be found within the lowest-most collective motion regimes. Further, we begin to see that there are slight differences in response behaviors dependent upon pulse interval times in the slightly higher frequency ranges.

The larger low-frequency response of non-allosteric residues compared to allosteric ones can be explained by considering the nature of allosteric communication and protein dynamics. Allosteric residues might preferentially participate in higher frequency, more localized motions that are crucial for signal transmission, while non-allosteric residues may be more involved in larger-scale, low-frequency motions. (44,45) These “mid-to-low” normal-mode frequencies have in fact been shown to act as unique dynamic signatures in differentiating biophysical behavior for different protein sequences within a given homologous family. (45) Allosteric residues may exhibit, generally, dampened low-frequency motions to maintain specificity in signal transmission, whereas non-allosteric residues may not have this constraint.

Even with cleanly separated results in the low-frequency ranges, it becomes apparent that additional analysis is required to make use of the full Fourier-transformed response profiles because they contain a large number of frequency components, especially when dealing with complex signals or datasets. Thus, we use PC analysis (PCA) to reduce this high-dimensional data into a lower-dimensional representation while retaining the most significant information. By identifying the PCs that capture the most variance in the data, PCA helps to compress and simplify the frequency space representation.

With this in mind, we next expand our dataset by including a total of 4 non-allosteric residues and 6 allosteric residues (40) in an attempt to leverage the dimensionality reduction of PCA with the information available through the frequency space response profiles to active site perturbations. Further, we expect that there should be similar distinct allosteric response patterns when other active sites are perturbed. Thus, in addition to active site S70 perturbations, we also include Fourier-transformed PCA analysis of perturbations to the other active sites K73, S130, E166, and K234. Here, we calculated perturbation response profiles with various pulse intervals, ranging from 0.25 to 20.75 ns with 0.25 ns intervals for a total of 83 response profiles per perturbed position. We determined the “best” pulse intervals by which the first two PCs could separate out allosteric from non-allosteric residues by using a support vector machine (SVM) to calculate the hyperplane distance separating the two groups and enabling us to identify the pulse intervals corresponding to the greatest hyperplane distance per position.

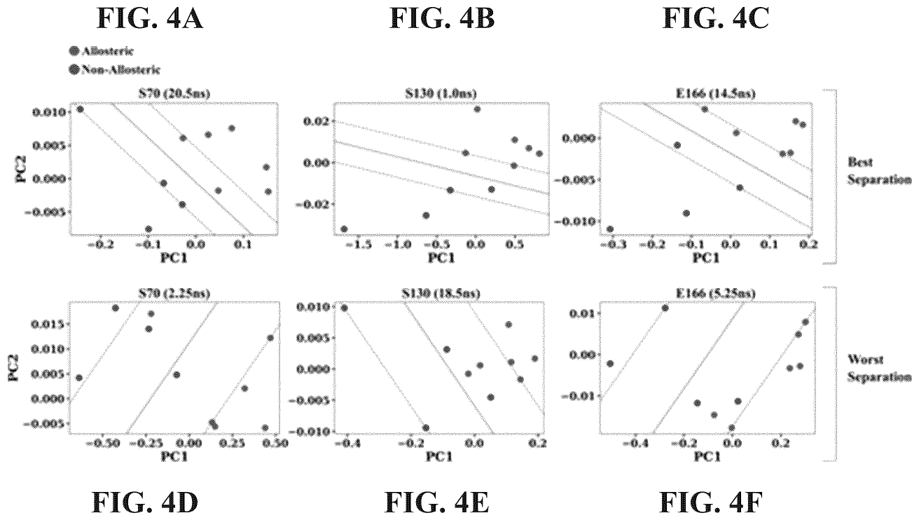

We find that, using only the first two PCs, it is possible to separate out allosteric from non-allosteric residues in this set of 10 positions (FIGS. 4A-4F). However, it becomes apparent that the variations in pulse intervals play a crucial role; only specific pulse intervals can generate the response profiles, establishing a clear division between allosteric and non-allosteric residues, while the other pulse intervals fail. Interestingly, the pulse interval between periodic force perturbations at a specific active site, which distinctly differentiates an allosteric response from a non-allosteric one, may not be effective for another active site. For example, while pulse intervals of 20.5 ns provided the most effective allosteric vs non-allosteric separation when applied to S70, pulse intervals of 1.0 ns yielded the best separation when applied to S130. This analysis highlights heterogeneous kinetics among allosteric sites and catalytic sites, where specific time scales govern the interaction between each catalytic site and the allosteric site.

### Time-Dependent Perturbation Response Profiles can Capture General Trends in Protein-Wide Substitution Behavior

Until this point, our work focused on distinguishing allosteric from non-allosteric residues within TEM-1, albeit at a limited scale; our final PCA covered only 10 total positions. Despite this limited set, our results show that this technique can effectively differentiate allosteric residues from other positions. This suggests that, to some extent, information about the unique contributions of each position to the overall interaction network of the protein is encoded within these time-dependent perturbation response profiles.

Consequently, we propose that it should be possible to capture the mutational impact of each position on the biophysical function, specifically mutations on the allosteric residues, while still restricting our analyses to the WT protein. This could potentially allow the characterization and identification of additional unknown allosteric positions without the need to perform computational simulations of all mutants of interest.

With this hypothesis in mind, we take advantage of the existing DMS data of the TEM-1 protein, (25) which provides comprehensive mutational data (covering all 20 amino acid types) for AMP and CTX fitness values at every position within TEM-1. In the case of AMP, fitness values were obtained at several concentration levels.

To investigate the relationship between the dynamic response of residues to active site perturbations and their impact on AMP resistance, we employed a multistep approach using time-dependent linear response theory. First, we individually perturbed each active site residue, utilizing pulse intervals aligned with the best allosteric separation from the analysis presented in FIGS. 4A-4F, and calculated the resulting response profiles for every position within the protein. We then Fourier-transformed these response profiles into frequency space and performed PCA of the transformed data. This process yielded PCs, clustering each position's response to a given active site perturbation.

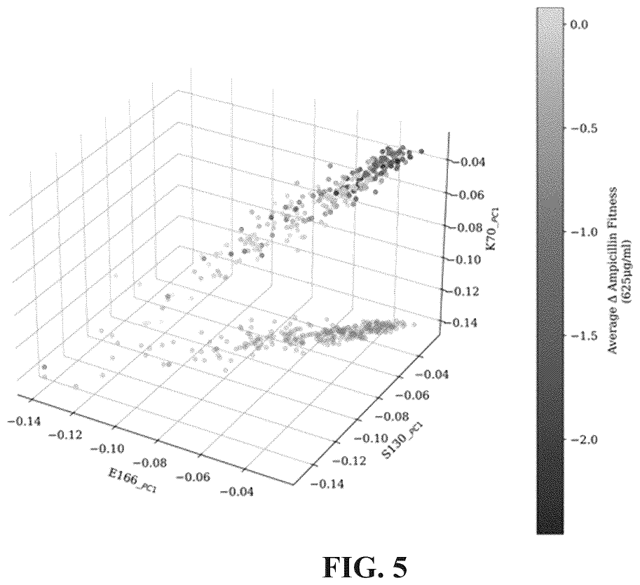

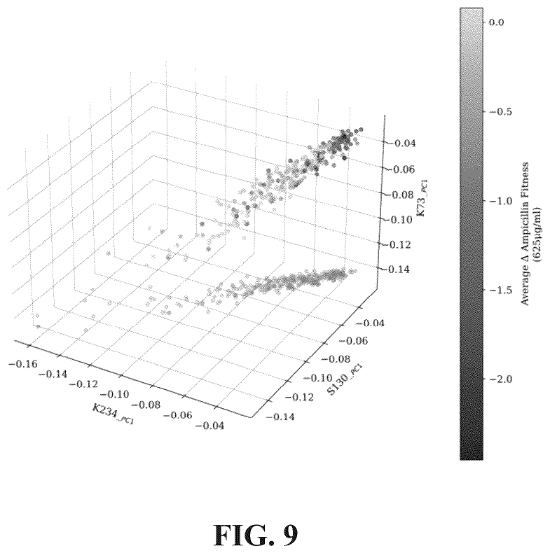

To visualize the relationship between the perturbation response and mutational impact on AMP resistance, we generated 3D plots using the first PC of perturbations to active sites S70, S130, and E166 for every position in the protein (FIG. 5). Each data point, corresponding to a specific residue position, was colored by the average change in AMP fitness values across all substitutions at that position, measured at 625 μg/mL. Notably, we observed a general trend between the first PC values and the average change in the AMP fitness for a given position. This trend was consistent regardless of the three active site perturbations used to calculate the PCs (see FIG. 9).

Classification Models Built on Time-Dependent Perturbation Responses can Identify a Given Position's Ability to Regulate AMP or CTX Resistance with High Accuracy

As shown above, while it is clear that associating Fourier-transformed PC values with the behavior of AMP fitness captures a general trend, the question still arises whether the time-dependent perturbation responses can be used to correctly identify specific individual positions that can contribute strongly to the antibacterial resistance.

Here, we attempted to train a logistic regression classification model with the goal of identifying positions that can strongly confer AMP or CTX resistance. To this end, we created binary classifications for each antibiotic based on their experimental data. For AMP, we used the fitness data associated with the 625 μg/mL concentration, averaged over two available experimental trials. Here, we iterated over all mutations per position, where mutations that resulted in change in fitness values (change in fitness from WT TEM-1, log fₘᵤₜ/f_{wt}) of ≥−0.2 were marked as 1 exhibiting neutrality similar to that of WT as penicillin degraders following the criteria in the original work, (25) else marked as 0 indicating a deleterious phenotype. The final average of these binarized values over 20 mutations per position was then taken, and positions with an average value of ≥0.5 were set to class=1, while all others were set to class=0. For CTX, mutations that could potentially increase CTX resistance were far less prevalent than in the case of AMP. To that end, if any mutation to a given position resulted in a CTX fitness value of ≥0.2, this position was set to class=1, while all others were set to class=0. As such, for AMP, positions of class=1 are those positions, which generally result in neutral-to-beneficial outcomes upon mutation. For CTX, positions of class=1 are those that may result in a putative beneficial mutation. Under this labeling system, out of 263 positions for which full coverage data was available, 159 positions were of class=1 for AMP, and 35 positions were of class=1 for CTX.

To estimate the probability that a given position belonged to class=1, we used a bootstrapping method with 1000 iterations of logistic regression, randomly splitting the data into 80:20 training/testing sets. Along with the goal of mutational outcome identification, we ultimately wanted to maintain these models with the goal of blind prediction for future work. To that end, after every iteration of logistic regression, the coefficients and intercept of the model fit to the data were extracted. Log-odds were calculated by summing the multiplication of positional feature values to their coefficient counterparts and adding the intercept value. The probabilities of a position being either AMP or CTX resistant (class=1) were then extracted by applying a sigmoidal function to these log-odds. Positions which had probabilities≥0.5 were considered positive, while all others were considered negative.

For potential features to include in our classification model, we used the first 50 PCs from perturbations to active sites S70, K73, S130, E166, and K234 as mentioned previously. Response profiles were generated with various pulse intervals, ranging from 0.25 to 20.75 ns with 0.25 ns intervals for a total of 83 response profiles per perturbed position. From these, we use the first 50 PCs from response profiles at each pulse interval for every perturbed position, resulting in a 20,750 total putative features.

In order to reduce the number of features used and improve the identification accuracy while avoiding an exhaustive search through the combinatorial feature space, we implemented logistic regression by considering one feature at a time. For each individual feature, we calculated true positive rates (TPR), false positive rates, true negative rates, false negative rates, and accuracy. The feature that yielded the highest TPR gain was selected and added to the logistic regression model's feature list. This iterative process was repeated by incorporating the previously identified feature along with one of the remaining 20,749 features, evaluating their performance one by one to identify the next best feature to include. The cycle continued until maximum TPRs were achieved for both AMP and CTX resistant classes using a specific set of features. To minimize the number of features, we refine the feature sets: initially starting with a set of k total features, we evaluated all subsets of (k−1) features. Any (k−1) subset that maintained the maximum accuracy associated with the original set of k features underwent the same iterative process, until we selected the minimum number of features that yielded the highest achievable accuracy.

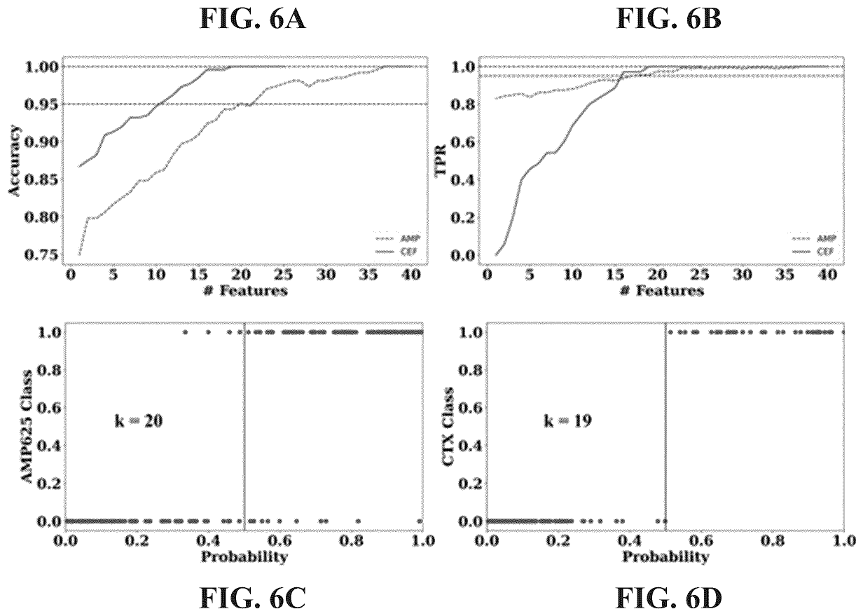

From this process, the associated accuracy and TPR per number of features are given in FIG. 6A, FIG. 6B respectively. We see that it is possible to correctly identify these positions with very high accuracy using a relatively limited number of features as compared to the 263 positions analyzed. In FIG. 6C, FIG. 6D, we show example cases probability values versus class for AMP and CTX models, with k=19 and 20, and accuracy=0.95 and 1.00, respectively, along with their feature breakdown by positions perturbed and pulse intervals.

Further analysis of the feature contributions to the logistic regression classification models associated with AMP and CTX resistance shows interesting behavior. Using only four features, our AMP classification model achieves 80% accuracy with 136 true positives and 28 false positives, and our CTX classification model achieves 90% accuracy with 14 true positives and 3 false positives.

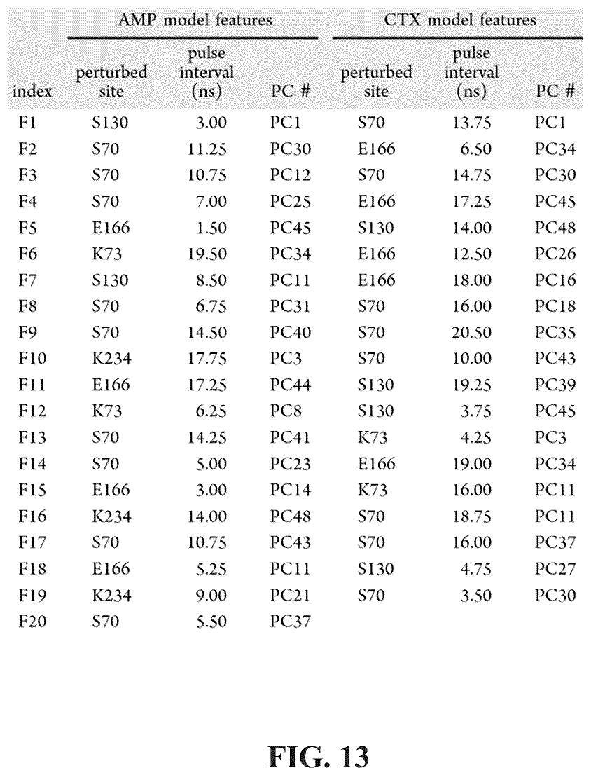

Inspection of FIG. 13 shows that three of these four features are responses to active site S70 perturbations and one is to active site S130 perturbations for AMP. For CTX, two of these four features are responses to active site S70 perturbations, and two are responses to active site E166 perturbations. In fact, four of the first seven features of the CTX classification model are E166 perturbation responses, whereas the AMP classification model only uses one E166 perturbation response in the first 10 features.

Interestingly, this behavior is in alignment with previous work, which suggested that the evolution of CTX hydrolysis starting from TEM-1 proceeded through coordinated alignment and positioning of a reactive C═O bond into the active site along with residues S70, E166, and reactive water. (46) Additionally, within the CTX-M class of β-lactamases, variants that increased CTX resistance also significantly impacted binding pocket atoms of position 166. (47) In both models, perturbations to position S70 played a significant role, comprising 9/20 features in the AMP classification model and 8/18 features in the CTX classification model.

Probabilities from Classification Models Can Correlate with AMP Minimum Values

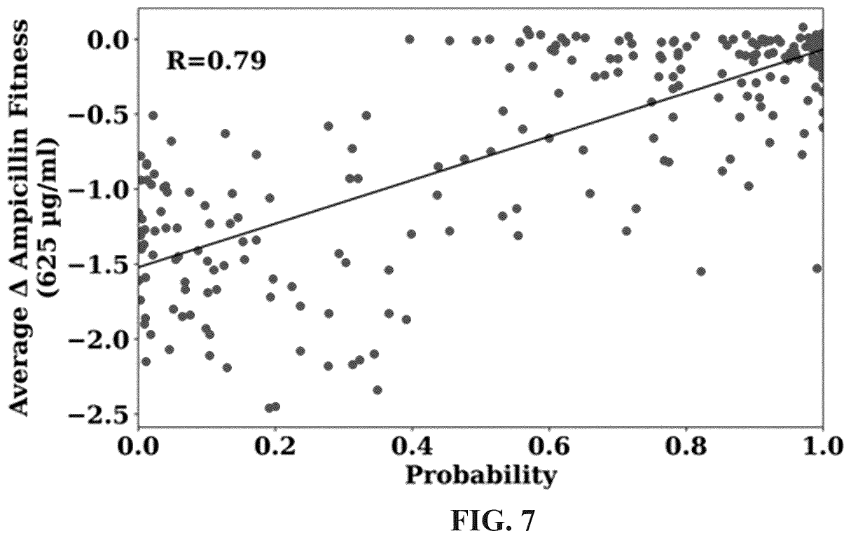

While our findings confirm that time-dependent response profiles can correctly identify if a position is likely susceptible to AMP or CTX degradation upon mutation through binary classification, it remains unclear as to whether this type of analysis can be extended further and relate to the continuum of fitness values associated with any given position's mutational landscape. To test this, we compared the probabilities from the logistic regression classification model for AMP to the average AMP fitness at 625 μg/mL concentration across all substitutions (FIG. 7). Here, a reasonably strong correlation exists with a Pearson R=0.79.

This suggests that even finer detail and more accurate correlations and identifications using this time-dependent perturbation analysis can be possible, as this correlation was made from binary classification probabilities. With more data and robust implementations of continuum prediction models such as modern neural network and AI architecture, it may be possible to fill in sparse experimental data with likely values using computation alone.

In this study, we have demonstrated the effectiveness of time-dependent perturbation response profiles in characterizing allosteric residues as well as the positional contribution to antibiotic resistance in the TEM-1 protein. Through our analysis, we successfully distinguished allosteric residues from non-allosteric ones and captured the unique information encoded within perturbation response profiles. Further, the logistic regression classification models achieved high accuracy in identifying positions that confer resistance to AMP and CTX. We observed that different active sites and pulse intervals played varying roles in the classification models, emphasizing the importance of considering specific antibiotic characteristics.

In future work, it would be valuable to explore the generalizability of our findings to other protein systems and determine whether the relationship between active sites holds across different proteins. Additionally, investigating the impact of longer perturbation intervals and relaxation times could provide further insights into the dynamics of the protein response profiles.

Furthermore, we anticipate that our time-dependent perturbation analysis can be extended to predict the continuum of fitness values associated with mutational landscapes. By incorporating more data and developing robust continuum prediction models integrating advanced machine learning techniques such as neural networks and AI architectures, we could enhance the accuracy and predictive power of our classification models to fill in experimental data gaps and make accurate predictions solely through computational approaches.

Overall, this study highlights the potential of time-dependent perturbation analysis as a valuable tool for unraveling protein dynamics and understanding drug resistance mechanisms. Our findings contribute to the field of allosteric regulation and pave the way for future investigations into allosteric residues and their role in antibiotic resistance. By expanding our knowledge in this area, we can ultimately develop more effective strategies for combating antibiotic resistance and improving drug development processes.

### Time-Dependent Linear Response Theory

Here we describe a framework to calculate the time-dependent response of residue i due to periodic force perturbations over the time in the protein. In our notations, we assume a coarse-grained approximation of the protein where each residue in the protein is represented by a bead. It should be noted that this framework is easily and directly transferrable to an all-atomic framework where i and j can represent specific atoms in the protein. We here show the formal derivation in 1-D as an example, where the generalization to the 3-D case follows exactly when taking appropriate vector products.

For the calculation of the response, assume equilibrium dynamics of the protein. This implies that the protein exists in a state of dynamic equilibrium with its surroundings where its phase space does not change over time. This condition for equilibrium can be expressed with the help of Liouville equation as:

$$\frac{\partial {ω}_{0}}{\partial t} = ( {H}_{0} , {ω}_{0} )$$

Here, the vector ωₒ represents the phase space, Hₒ is the Hamiltonian at equilibrium for the protein and “( )” represents the Poisson backets. Under these conditions, if a force, f(t) is applied, the time average response at residue position i, at time t can be calculated as:

$$〈 Δ {r}_{i} ( t ) 〉 = {\int }_{0}^{t} {dt}^{′} ϕ ( t - {t}^{′} ) f ( {t}^{′} )$$

Where φ is the response function. Another way to re-write the time average of response is by integrating over the phase space of the protein, i.e.,

$$〈 Δ {r}_{i} ( t ) 〉 = 〈 Δ {r}_{i} ( t ) \vert ω ( t ) 〉$$

Where operator bracket <A|B> is the inner product between A and B.

To calculate the response function, we use perturbation theory where we calculate the impact of perturbation on the equilibrium phase space of the protein. According to Liouville equation, the response to perturbation on the phase space can be calculated as:

$$\frac{\partial ω ( t )}{\partial t} = ( {H}_{f} , ω ( t ) )$$

Where H is the Hamiltonian of the perturbed protein with m particles where each particle j has an external force fⱼ(t) exerted on them:

$${H}_{f} = {H}_{0} - {\sum }_{j = 1}^{m} Δ {r}_{j} {f}_{j} ( t )$$

And ω(t) is the perturbed phase space with the response Δω(t):

$$ω ( f ) = {ω}_{0} + Δω ( t )$$

Inserting the expression for H_{f} and ω(t) into the Liouville equation, we get:

$$\frac{\partial ( {ω}_{0} + Δ ω ( t ) )}{\partial t} = ( {H}_{0} − {\sum }_{j = 1}^{m} Δ {r}_{j} {f}_{j} ( t ) , {ω}_{0} + Δ ω ( t ) )$$

Since Poisson brackets associate and using equation A,

$$\frac{\partial Δω ( t )}{\partial t} = ( {H}_{0} Δω ( t ) ) - ( {\sum }_{j = 1}^{m} {Δ r}_{j} {f}_{j} ( t ) , {ω}_{0} ) - ( {\sum }_{j = 1}^{m} {Δ r}_{j} {f}_{j} ( t ) , Δω ( t ) )$$

Here, the middle term in the expression is vanishingly close to zero for studying small perturbations in linear response limit. Eliminating that term, we can rewrite this as:

$$\frac{\partial Δω ( t )}{\partial t} = {iL}_{o} Δω ( t ) - {\sum }_{j = 1}^{m} {f}_{j} ( t ) ( {Δ r}_{j} , Δω ( t ) )$$

Where Lₒ is the Liouville operator which describes the evolution of perturbed phase space under an equilibrium Hamiltonian. The solution of this equation is of the form: Δω(t)=C(t)exp(itLₒ). Feeding this solution back into the first term on the right-hand side of the equation above,

$$\frac{\partial Δω ( t )}{\partial t} = {iL}_{o} C ( t ) {e}^{{itL}_{o}} - {\sum }_{j = 1}^{m} {f}_{j} ( t ) ( {Δ r}_{j} , Δω ( t ) ) and also \qquad ( C )$$

$$\frac{\partial Δω ( t )}{\partial t} = {iL}_{o} C ( t ) {e}^{{itL}_{o}} - \frac{\partial }{\partial t} C ( t ) {e}^{{itL}_{o}}$$

Comparing the two equations (B and C), we get

$$\frac{\partial }{\partial t} C ( t ) {e}^{{itL}_{o}} = - {\sum }_{j = 1}^{m} {f}_{j} ( t ) ( {Δ r}_{j} , Δω ( t ) )$$

$$\frac{\partial C ( t )}{\partial t} = - {\sum }_{j = 1}^{m} {e}^{- {itL}_{o}} {f}_{j} ( t ) ( {Δ r}_{j} , Δω ( t ) )$$

$$C ( t ) = - {\int }_{0}^{t} {e}^{- {iL}_{o} ( {t}^{′} - t )} {\sum }_{j = 1}^{m} {f}_{j} ( {t}^{′} ) ( {Δ r}_{j} , Δω ( {t}^{′} ) ) {dt}^{′}$$

Here we should note that at t=0, the system is in equilibrium, i.e., Δω(0)=0 therefore, C(0)=0.

$$Δω ( t ) = - {\int }_{0}^{t} {e}^{- {it}^{′} {L}_{o} ( {t}^{1} - t )} {\sum }_{j = 1}^{m} ( {Δ r}_{j} , {Δω}_{o} ) {f}_{j} ( {t}^{′} ) {e}^{- {itL}_{o}} {dt}^{′}$$

$$= {\int }_{0}^{t} {e}^{- i ( {t}^{′} - t ) {L}_{o}} {\sum }_{j = 1}^{m} ( {Δ r}_{j} , {Δω}_{o} ) {f}_{j} ( {t}^{′} ) {dt}^{′}$$

Going back to equation A2, we can re-write it as:

$$〈 {Δ r}_{i} ( t ) 〉 = 〈 {Δ r}_{i} ( t ) \vert {ω}_{0} 〉 + 〈 {Δ r}_{i} ( t ) \vert Δω ( t ) 〉$$

Here the first term reduces to 0 as it describes the average fluctuation in residue j at equilibrium.

$$〈 {Δ r}_{i} ( t ) 〉 = 〈 {Δ r}_{i} ( t ) \vert Δω ( t ) 〉$$

$$= \int \int {dpdq Δ r}_{i} ( t ) Δω ( t )$$

Substituting in from Equation D,

$$〈 {Δ r}_{i} ( t ) 〉 = {\int }_{0}^{t} {dt}^{′} \int \int {dpdq Δ r}_{i} ( {t}^{′} ) {e}^{- i ( {t}^{′} - t ) {L}_{o}} {\sum }_{j = 1}^{m} ( {Δ r}_{j} , {Δω}_{o} ) {f}_{j} ( {t}^{′} ) \qquad ( D1 )$$

$$= - {\int }_{0}^{t} {dt}^{′} \int \int dpdq {\sum }_{j = 1}^{m} ( {ω}_{o} , {Δ r}_{j} ( {t}^{′} ) ) {Δ r}_{i} ( t - {t}^{′} ) {f}_{j} ( {t}^{′} )$$

$$= {\int }_{0}^{t} {dt}^{′} ϕ ( t - {t}^{′} ) {f}_{j} ( {t}^{′} )$$

where, −Δr(t−t′)=e^{-i (t′-t)Lo}Δr as Lₒ gives the time evolution of the observable (the fluctuation of residue i) and the φ is the effective response function. The double integral over the generalized coordinates and momentum, dpdq, is the standard notation for integrating over phase space. Therefore,

$$ϕ ( t ) = - \int \int dpdq {\sum }_{j = 1}^{m} ( {ω}_{o} , {Δ r}_{j} ( 0 ) ) {Δ r}_{i} ( t ) \qquad ( D2 )$$

For any observable (here Δrⱼ(0)), we can write

$$Δ {\dot{r}}_{j} ( 0 ) = ( {H}_{o} , {Δ r}_{j} ( 0 ) ) \qquad ( E )$$

Also, the equilibrium Hamiltonian Hₒ and the phase space ωₒ are related to each other as,

$${ω}_{0} = \frac{{e}^{- {β H}_{o}}}{\int \int {e}^{- {β H}_{o}} dpdq}$$

$$= \frac{{e}^{- {β H}_{o}}}{Q}$$

$$- {β H}_{o} = ln ( {Q ω}_{0} )$$

$$= ln ( Q ) + ln ( {ω}_{0} )$$

Where β=1/k_{B}T with k_{B} as the Boltzmann Constant and Q is a constant for a given system under equilibrium is called the Partition Function. Using this relationship in Equation E,

$$Δ {\dot{r}}_{j} ( 0 ) = ( {H}_{o} , {Δ r}_{j} ( 0 ) )$$

$$= - \frac{1}{β} ( ln ( Q ) + ln ( {ω}_{o} ) , {Δ r}_{j} ( 0 ) )$$

$$Δ {\dot{r}}_{j} ( 0 ) = - \frac{1}{β} ( ln ( {ω}_{o} ) , {Δ r}_{j} ( 0 ) )$$

$$= - \frac{1}{{βω}_{o}} ( {ω}_{o} , {Δ r}_{j} ( 0 ) )$$

Using this, the response function from equation D2 can be re-written as:

$$ϕ ( t ) = \int \int dpdq {\sum }_{j = 1}^{m} ( βΔ {\dot{r}}_{j} ( 0 ) ) {Δ r}_{i} ( t )$$

$$= β {\sum }_{j = 1}^{m} {〈 Δ {\dot{r}}_{j} ( 0 ) {Δ r}_{i} ( t ) 〉}_{0}$$

Here, the covariance between the velocity of residue j at time 0 with the displacement of residue i is calculated under the conditions of the equilibrium. Finally, the response of the system to perturbations, in equation D1, can be expressed as:

$$〈 {Δ r}_{i} ( t ) 〉 = \frac{1}{{k}_{B} T} {\int }_{0}^{t} {dt}^{′} {\sum }_{j = 1}^{m} {〈 Δ {\dot{r}}_{j} ( 0 ) {Δ r}_{i} ( {t}^{′} ) 〉}_{0} {f}_{j} ( t - {t}^{′} )$$

In order to calculate the response, we can use the trajectory of a protein obtained from molecular dynamic simulations. For the work presented here, 50 perturbations of unit force in randomized direction were applied at a given pulse interval to a single residue j at a time and the average responses over these 50 perturbations were calculated. In cases for only one single perturbed residue j, we have our fluctuation response of residue i as:

$$〈 {Δ r}_{i} ( t ) 〉 = \frac{1}{{k}_{B} T} {\int }_{0}^{t} {dt}^{′} {〈 Δ {\dot{r}}_{j} ( 0 ) {Δ r}_{i} ( {t}^{′} ) 〉}_{0} {f}_{j} ( t - {t}^{′} ) .$$

### REFERENCES FOR EXAMPLE 1

1. Campitelli, P.; Ozkan, S. B. Allostery and Epistasis: Emergent Properties of Anisotropic Networks. Entropy 2020, 22 (6), 667, DOI: 10.3390/e22060667
2. Campitelli, P.; Modi, T.; Kumar, S.; Ozkan, S. B. The Role of Conformational Dynamics and Allostery in Modulating Protein Evolution. Annu. Rev. Biophys. 2020, 49, 267-288, DOI: 10.1146/annurev-biophys-052118-115517
3. Campitelli, P.; Guo, J.; Zhou, H.-X.; Ozkan, S. B. Hinge-Shift Mechanism Modulates Allosteric Regulations in Human Pin1. J. Phys. Chem. B 2018, 122 (21), 5623-5629, DOI: 10.1021/acs.jpcb.7b11971
4. Campitelli, P.; Swint-Kruse, L.; Ozkan, S. B. Substitutions at Nonconserved Rheostat Positions Modulate Function by Rewiring Long-Range, Dynamic Interactions. Mol. Biol. Evol. 2021, 38 (1), 201-214, DOI: 10.1093/molbev/msaa202.
5. Campitelli, P.; Lu, J.; Ozkan, S. B. Dynamic allostery highlights the evolutionary differences between the CoV-1 and CoV-2 main proteases. Biophys. J. 2022, 121 (8), 1483-1492, DOI: 10.1016/j.bpj.2022.03.012.
6. Deng, J.; Yuan, Y.; Cui, Q. Modulation of Allostery with Multiple Mechanisms by Hotspot Mutations in TetR. J. Am. Chem. Soc. 2024, 146 (4), 2757-2768, DOI: 10.1021/jacs.3c12494.
7. Gerek, Z. N.; Ozkan, S. B. Change in allosteric network affects binding affinities of PDZ domains: Analysis through perturbation response scanning. PLOS Comput. Biol. 2011, 7 (10), e1002154 DOI: 10.1371/journal.pcbi. 1002154.
8. Guo, J.; Pang, X.; Zhou, H.-X. Two Pathways Mediate Inter-Domain Allosteric Regulation in Pin1. Structure 2015, 23 (1), 237-247, DOI: 10.1016/j.str.2014.11.009.
9. Kazan, I. C.; Mills, J. H.; Ozkan, S. B. Allosteric regulatory control in dihydrofolate reductase is revealed by dynamic asymmetry. Protein Sci. 2023, 32 (8), e4700 DOI: 10.1002/pro.4700.
10. Nussinov, R.; Tsai, C.-J. Allostery in disease and in drug discovery. Cell 2013, 153 (2), 293-305, DOI: 10.1016/j.cell.2013.03.034.
11. Ose, N. J.; Butler, B. M.; Kumar, A.; Kazan, I. C.; Sanderford, M.; Kumar, S.; Ozkan, S. B. Dynamic coupling of residues within proteins as a mechanistic foundation of many enigmatic pathogenic missense variants. PLOS Comput. Biol. 2022, 18 (4), e1010006 DOI: 10.1371/journal.pcbi.1010006.
12. May, L. T.; Leach, K.; Sexton, P. M.; Christopoulos, A. Allosteric modulation of G protein-coupled receptors. Annu. Rev. Pharmacol. Toxicol. 2007, 47, 1-51, DOI: 10.1146/annurev.pharmtox.47.120505.105159
13. Nussinov, R.; Tsai, C.-J. The Different Ways through Which Specificity Works in Orthosteric and Allosteric Drugs. Curr. Drug Metab. 2012, 18 (9), 1311-1316, DOI: 10.2174/138920012799362855.
14. Wenthur, C. J.; Gentry, P. R.; Mathews, T. P.; Lindsley, C. W. Drugs for allosteric sites on receptors. Annu. Rev. Pharmacol. Toxicol. 2014, 54, 165-184, DOI: 10.1146/annurev-pharmtox-010611-134525.
15. Wagner, J. R.; Lee, C. T.; Durrant, J. D.; Malmstrom, R. D.; Feher, V. A.; Amaro, R. E. Emerging Computational Methods for the Rational Discovery of Allosteric Drugs. Chem. Rev. 2016, 116 (11), 6370-6390, DOI: 10.1021/acs.chemrev.5b00631.
16. Wang, J.; Jain, A.; McDonald, L. R.; Gambogi, C.; Lee, A. L.; Dokholyan, N. V. Mapping allosteric communications within individual proteins. Nat. Commun. 2020, 11 (1), 3862, DOI: 10.1038/s41467-020-17618-2.
17. Molgedey, L.; Schuster, H. G. Separation of a mixture of independent signals using time delayed correlations. Phys. Rev. Lett. 1994, 72 (23), 3634-3637, DOI: 10.1103/PhysRevLett.72.3634.
18. Naritomi, Y.; Fuchigami, S. Slow dynamics of a protein backbone in molecular dynamics simulation revealed by time-structure based independent component analysis. J. Chem. Phys. 2013, 139 (21), 215102, DOI: 10.1063/1.4834695.
19. Schultze, S.; Grubmüller, H. Time-Lagged Independent Component Analysis of Random Walks and Protein Dynamics. J. Chem. Theory Comput. 2021, 17 (9), 5766-5776, DOI: 10.1021/acs.jctc.1c00273.
20. Ose, N. J.; Campitelli, P.; Modi, T.; Kazan, I. C.; Kumar, S.; Ozkan, S. B. Some mechanistic underpinnings of molecular adaptations of SARS-COV-2 spike protein by integrating candidate adaptive polymorphisms with protein dynamics. bioRxiv 2023, 12, RP92063, DOI: 10.1101/2023.09.14.557827.
21. Modi, T.; Ozkan, S. B. Mutations Utilize Dynamic Allostery to Confer Resistance in TEM-1 β-lactamase. Int. J. Mol. Sci. 2018, 19 (12), 3808, DOI: 10.3390/ijms19123808.
22. Yang, L.-W.; Kitao, A.; Huang, B.-C.; Gō, N. Ligand-induced protein responses and mechanical signal propagation described by linear response theories. Biophys. J. 2014, 107 (6), 1415-1425, DOI: 10.1016/j.bpj.2014.07.049.
23. Huang, B.-C.; Yang, L.-W. Molecular dynamics simulations and linear response theories jointly describe biphasic responses of myoglobin relaxation and reveal evolutionarily conserved frequent communicators. Biophys. Physicobiol. 2019, 16, 473-484, DOI: 10.2142/biophysico.16.0_473.
24. Fonzé, E.; Charlier, P.; To'th, Y.; Vermeire, M.; Raquet, X.; Dubus, A.; Frère, J. M. TEM1 β-lactamase structure solved by molecular replacement and refined structure of the S235A mutant. Acta Crystallogr., Sect. D: Biol. Crystallogr. 1995, 51 (5), 682-694, DOI: 10.1107/S0907444994014496.
25. Stiffler, M. A.; Hekstra, D. R.; Ranganathan, R. Evolvability as a Function of Purifying Selection in TEM-1 β-Lactamase. Cell 2015, 160 (5), 882-892, DOI: 10.1016/j.cell.2015.01.035.
26. Knies, J. L.; Cai, F.; Weinreich, D. M. Enzyme efficiency but not thermostability drives cefotaxime resistance evolution in TEM-1 β-lactamase. Mol. Biol.

Evol. 2017, 34 (5), 1040-1054, DOI: 10.1093/molbev/msx053.
27. Jacquier, H.; Birgy, A.; Le Nagard, H.; Mechulam, Y.; Schmitt, E.; Glodt, J.; Bercot, B.; Petit, E.; Poulain, J.; Barnaud, G.; Gros, P.-A.; Tenaillon, O. Capturing the mutational landscape of the beta-lactamase TEM-1. Proc. Natl. Acad. Sci. U.S.A. 2013, 110 (32), 13067-13072, DOI: 10.1073/pnas. 1215206110.
28. Figliuzzi, M.; Jacquier, H.; Schug, A.; Tenaillon, O.; Weigt, M. Coevolutionary Landscape Inference and the Context-Dependence of Mutations in Beta-Lactamase TEM-1. Mol. Biol. Evol. 2016, 33 (1), 268-280, DOI: 10.1093/molbev/msv211.
29. Alvarez, S.; Nartey, C. M.; Mercado, N.; de la Paz, J. A.; Huseinbegovic, T.; Morcos, F. In vivo functional phenotypes from a computational epistatic model of evolution. Proc. Natl. Acad. Sci. U.S.A. 2024, 121 (6), e2308895121 DOI: 10.1073/pnas.2308895121.
30. Gonzalez, C. E.; Roberts, P.; Ostermeier, M. Fitness Effects of Single Amino Acid Insertions and Deletions in TEM-1 β-Lactamase. J. Mol. Biol. 2019, 431 (12), 2320-2330, DOI: 10.1016/j.jmb.2019.04.030.
31. Salverda, M. L. M.; De Visser, J. A. G.; Barlow, M. Natural evolution of TEM-1 β-lactamase: experimental reconstruction and clinical relevance. FEMS Microbiol. Rev. 2010, 34 (6), 1015-1036, DOI: 10.1111/j.1574-6976.2010.00222.x.
32. Case, D. A, et al., A. Amber 2020; University of California, San Francisco, 2020.
33. Jelsch, C.; Mourey, L.; Masson, J. M.; Samama, J. P. Crystal structure of Escherichia coli TEM1 β-lactamase at 1.8 Å resolution. Proteins 1993, 16 (4), 364-383, DOI: 10.1002/prot.340160406.
34. Mackerell, A. D.; et al. All-atom empirical potential for molecular modeling and dynamics studies of proteins. J. Phys. Chem. B 1998, 102 (18), 3586-3616, DOI: 10.1021/jp973084f
35. Ryckaert, J.-P.; Ciccotti, G.; Berendsen, H. J. Numerical integration of the cartesian equations of motion of a system with constraints: molecular dynamics of n-alkanes. J. Comput. Phys. 1977, 23 (3), 327-341, DOI: 10.1016/0021-9991 (77) 90098-5.
36. Darden, T.; York, D.; Pedersen, L. Particle mesh Ewald: An N·log (N) method for Ewald sums in large systems. EMBO J. 1993, 98 (12), 10089-10092, DOI: 10.1063/1.464397.
37. Butler, B. M.; Gerek, Z. N.; Kumar, S.; Ozkan, S. B. Conformational dynamics of nonsynonymous variants at protein interfaces reveals disease association. Proteins 2015, 83 (3), 428-435, DOI: 10.1002/prot.24748.
38. Butler, B. M.; Kazan, I. C.; Kumar, A.; Ozkan, S. B. Coevolving residues inform protein dynamics profiles and disease susceptibility of nSNVs. PLOS Comput. Biol. 2018, 14 (11), e1006626 DOI: 10.1371/journal.pcbi. 1006626
39. Ose, N. J.; Campitelli, P.; Patel, R.; Kumar, S.; Ozkan, S. B. Protein dynamics provide mechanistic insights about epistasis among common missense polymorphisms. Biophys. J. 2023, 122 (14), 2938-2947, DOI: 10.1016/j.bpj.2023.01.037.
40. Bowman, G. R.; Bolin, E. R.; Hart, K. M.; Maguire, B. C.; Marqusee, S. Discovery of multiple hidden allosteric sites by combining Markov state models and experiments. Proc. Natl. Acad. Sci. U.S.A. 2015, 112 (9), 2734-2739, DOI: 10.1073/pnas. 1417811112.
41. Holliday, M. J.; Camilloni, C.; Armstrong, G. S.; Vendruscolo, M.; Eisenmesser, E. Z. Networks of Dynamic Allostery Regulate Enzyme Function. Structure 2017, 25 (2), 276-286, DOI: 10.1016/j.str.2016.12.003.
42. Xie, J.; Zhang, W.; Zhu, X.; Deng, M.; Lai, L. Coevolution-based prediction of key allosteric residues for protein function regulation. eLife 2023, 12, e81850 DOI: 10.7554/eLife.81850.
43. Bozovic, O.; Zanobini, C.; Gulzar, A.; Jankovic, B.; Buhrke, D.; Post, M.; Wolf, S.; Stock, G.; Hamm, P. Real-time observation of ligand-induced allosteric transitions in a PDZ domain. Proc. Natl. Acad. Sci. U.S.A. 2020, 117 (42), 26031-26039, DOI: 10.1073/pnas.2012999117.
44. Otten, R.; Pádua, R. A. P.; Bunzel, H. A.; Nguyen, V.; Pitsawong, W.; Patterson, M.; Sui, S.; Perry, S. L.; Cohen, A. E.; Hilvert, D.; Kern, D. How directed evolution reshapes the energy landscape in an enzyme to boost catalysis. Science 2020, 370 (6523), 1442-1446, DOI: 10.1126/science.abd3623.
45. Nussinov, R.; Tsai, C.-J.; Liu, J. Principles of allosteric interactions in cell signaling. J. Am. Chem. Soc. 2014, 136 (51), 17692-17701, DOI: 10.1021/ja510028c.
46. Schneider, S. H.; Kozuch, J.; Boxer, S. G. The Interplay of Electrostatics and Chemical Positioning in the Evolution of Antibiotic Resistance in TEM β-Lactamases. ACS Cent. Sci. 2021, 7 (12), 1996-2008, DOI: 10.1021/acscentsci.1c00880.
47. Latallo, M. J.; Cortina, G. A.; Faham, S.; Nakamoto, R. K.; Kasson, P. M. Predicting allosteric mutants that increase activity of a major antibiotic resistance enzyme. Chem. Sci. 2017, 8 (9), 6484-6492, DOI: 10.1039/C7SC02676E.

### Example 2: Illustrative Embodiments of Presented Methods

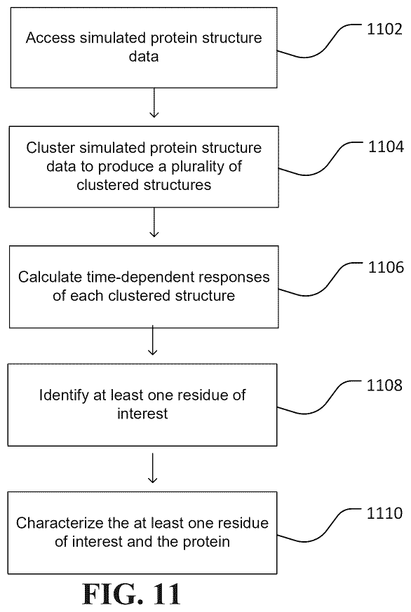

FIG. 11 shows an example process 1100 for characterizing a protein. Characterizing a protein may include: accessing simulated protein structure data at 1102. The simulated protein structure data may indicate a structure of the protein. At 1104, the method may include clustering the simulated protein structure data to produce a plurality of clustered structures and determining an equilibrium of each clustered structure in the plurality of clustered structures. At 1106, the method may include calculating time-dependent responses of each clustered structure int eh plurality of clustered structures. At 1108, the method may include identifying at least one residue of interest based on the time-dependent response of each clustered structure in the plurality of clustered structures. At 1110, the method may include characterizing the protein and the at least one residue of interest. The process 1100 may be executed on a computer system. The process 1100 may further include generating a report, and providing the report to a user.

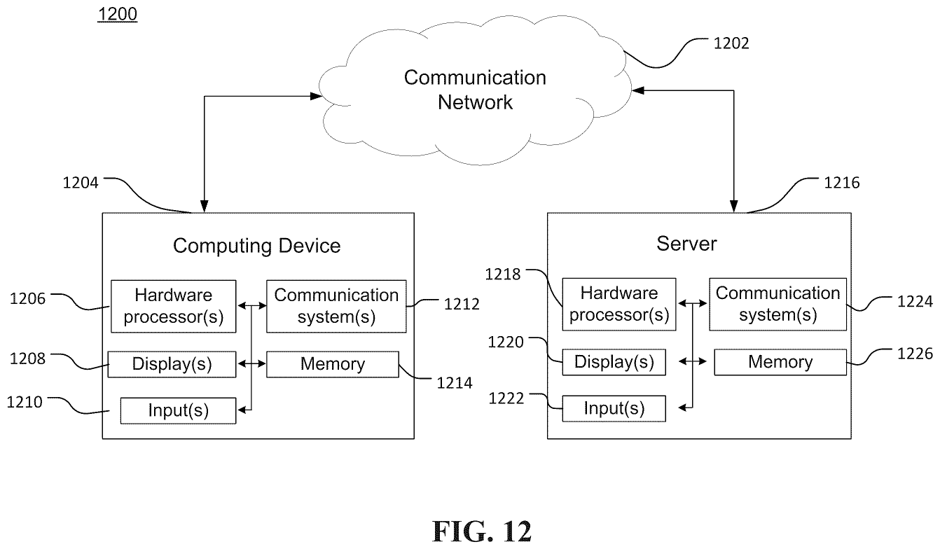

In FIG. 12, an example 1200 of a system (e.g., a data processing system) in accordance with some embodiments of the disclosed subject matter is shown.

In some embodiments, computing device 1204 and/or server 1216 can be any suitable computing device or combination of devices, such as a desktop computer, a laptop computer, a smartphone, a tablet computer, a wearable computer, a server computer, a virtual machine being executed by a physical computing device, etc. As described herein, system 1200 can present information about a characterized protein to a user (e.g., a researcher and/or a physician).

In some embodiments, communication network 1202 can be any suitable communication network or combination of communication networks. In some embodiments, communication network 1202 can be any suitable communication network or combination of communication networks. For example, communication network 1202 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 1202 can be a local area network, a wide area network, a public network (e.g., the Internet), a private or semi-private network (e.g., a corporate or university intranet), any other suitable type of network, or any suitable combination of networks. Communications links shown in FIG. 12 can each be any suitable communications link or combination of communications links, such as wired links, fiber optic links, Wi-Fi links, Bluetooth links, cellular links, etc.

FIG. 12 additionally shows an example of hardware that can be used to implement computing device 1204 and server 1216 in accordance with some embodiments of the disclosed subject matter. In some embodiments, computing device 1204 can be used to execute one or more set of instructions to characterize a protein. In other embodiments, computing device 1204 can be used to identify and characterize one or more residues of interest. In still other embodiments, computing device 1204 can be used to selectively mutate a protein, and compare the characterization of the non-mutated protein and the selectively mutated protein.

As shown in FIG. 12, computing device 1204 can include one or more hardware processor 1206, one or more displays 1208, one or more inputs 1210, one or more communications 1212, and/or memory 1214. In some embodiments, processor 1206 can be any suitable hardware processor or combination of processors, such as central processing unit, a graphics processing unit, etc. In some embodiments, display 1208 can include any suitable display devices, such as a computer monitor, a touchscreen, a television, etc. In some embodiments, inputs 1210 can include any suitable input device and/or sensors that can be used to receive user input, such as a keyboard, a mouse, a touchscreen, a microphone, etc.

In some embodiments, communication systems 1212 can include any suitable hardware, firmware, and/or software for communicating information over communication network 1202 and/or any other suitable communication networks. For example, communications systems 1212 can include one or more transceivers, one or more communication chips and/or chip sets, etc. In a more particular example, communications systems 1212 can include hardware, firmware and/or software that can be used to establish a Wi-Fi connection, a Bluetooth connection, a cellular connection, an Ethernet connection, etc.

In some embodiments, memory 1214 can include any suitable storage device or devices that can be used to store instructions, values, etc., that can be used, for example, by processor 1206 to present content using display 1208, to communicate with server 1216 via communications system(s) 1212, etc.

Memory 1214 can include any suitable volatile memory, non-volatile memory, storage, or any suitable combination thereof. For example, memory 1214 can include RAM, ROM, EEPROM, one or more flash drives, one or more hard disks, one or more solid state drives, one or more optical drives, etc. In some embodiments, memory 1214 can have encoded thereon a computer program for controlling operation of computing device 1204. In such embodiments, processor 1206 can execute at least a portion of the computer program to present content (e.g., images, user interfaces, graphics, tables, etc.), receive content from server 1216, transmit information to server 1216, etc.

In some embodiments, server 1216 can include a processor 1218, a display 1220, one or more inputs 1222, one or more communications systems 1224, and/or memory 1226. In some embodiments, processor 1218 can be any suitable hardware processor or combination of processors, such as a central processing unit, a graphics processing unit, etc. In some embodiments, display 1220 can include any suitable display devices, such as a computer monitor, a touchscreen, a television, etc. In some embodiments, inputs 1222 can include any suitable input devices and/or sensors that can be used to receive user input, such as a keyboard, a mouse, a touchscreen, a microphone, etc.

In some embodiments, communications systems 1224 can include any suitable hardware, firmware, and/or software for communicating information over communication network 1202 and/or any other suitable communication networks. For example, communications systems 1224 can include one or more transceivers, one or more communication chips and/or chip sets, etc. In a more particular example, communications systems 1224 can include hardware, firmware and/or software that can be used to establish a Wi-Fi connection, a Bluetooth connection, a cellular connection, an Ethernet connection, etc.

In some embodiments, memory 1226 can include any suitable storage device or devices that can be used to store instructions, values, etc., that can be used, for example, by processor 1218 to present content using display 1220, to communicate with one or more computing devices 1204, etc. Memory 1226 can include any suitable volatile memory, non-volatile memory, storage, or any suitable combination thereof. For example, memory 1226 can include RAM, ROM, EEPROM, one or more flash drives, one or more hard disks, one or more solid state drives, one or more optical drives, etc. In some embodiments, memory 1226 can have encoded thereon a server program for controlling operation of server 1216. In such embodiments, processor 1218 can execute at least a portion of the server program to transmit information and/or content (e.g., results of a tissue identification and/or classification, a user interface, etc.) to one or more computing devices 1204, receive information and/or content from one or more computing devices 1204, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

A number of references to patent and non-patent documents are made throughout the publication, each of which is herein incorporated by reference in its entirety.

While the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A method of characterizing a protein comprising:
accessing simulated protein structure data with a computer system, wherein the simulated protein structure data indicate a structure of the protein;
using the computer system to:
a) cluster the simulated protein structure data to produce a plurality of clustered structures and determine an equilibrium of each clustered structure in the plurality of clustered structures;
b) calculate time-dependent responses of each clustered structure in the plurality of clustered structures;
c) identify at least one residue of interest based on the time-dependent responses of each clustered structure in the plurality of clustered structures; and
d) characterize the at least one residue of interest and the protein.

2. The method of claim 1, wherein accessing the simulated protein structure data comprises simulating the structure of the protein with the computer system using isobaric canonical ensemble MD simulations.

3. The method of claim 1, wherein calculating the time-dependent responses of each clustered structure in the plurality of clustered structures comprises:
extracting cross velocity-positional covariance matrices corresponding to each clustered structure in the plurality of clustered structures; and
applying perturbative forces to the cross velocity-positional covariance matrices corresponding to each structure in the plurality of clustered structures to calculate the time-dependent responses.

4. The method of claim 1, wherein identifying the at least one residue of interest comprises using frequency domain analysis.

5. The method of claim 1, wherein identifying and characterizing the at least one residue of interest comprises identifying the at least one residue as allosteric or non-allosteric.

6. The method of claim 1, wherein the method is used to predict a mutational impact on the protein.

7. The method of claim 6, wherein predicting the mutational impact on the protein comprises:
selectively mutating the protein using single amino acid substitutions;
repeating steps (a)-(d) on the selectively mutated protein; and
comparing the characterization of the selectively mutated protein and the protein from step a.

8. The method of claim 1, wherein the method is used to investigate antibiotic resistance.

9. The method of claim 1, wherein the method further comprises generating a report comprising the characterization of the at least one residue and the protein.

10. A system for characterizing a protein comprising:
a non-transitory computer-readable medium having stored thereon instructions that, when executed by a processor, cause the processor to:
a) access simulated protein structure data, wherein the simulated protein structure data indicate a structure of the protein;
b) cluster the simulated protein structure data to produce a plurality of clustered structures and determine an equilibrium of each clustered structure in the plurality of clustered structures;
c) calculate time-dependent responses of each clustered structure in the plurality of clustered structures;
d) identify at least one residue of interest based on the time-dependent responses of each clustered structure in the plurality of clustered structures; and
e) characterize the at least one residue of interest and the protein.

11. The system of claim 10, wherein accessing the simulated protein structure data comprises simulating the structure of the protein with the computer system using isobaric canonical ensemble MD simulations.

12. The system of claim 10, wherein calculating the time-dependent responses of each clustered structure in the plurality of clustered structures comprises:
extracting cross velocity-positional covariance matrices corresponding to each clustered structure in the plurality of clustered structures; and
applying perturbative forces to the cross velocity-positional covariance matrices corresponding to each structure in the plurality of clustered structures to calculate the time-dependent responses.

13. The system of claim 10, wherein identifying the at least one residue of interest comprises using frequency domain analysis.

14. The system of claim 10, wherein identifying and characterizing the at least one residue of interest comprises identifying the at least one residue as allosteric or non-allosteric.

15. The system of claim 10, wherein the system is used to predict a mutational impact on the protein.

16. The system of claim 10, wherein predicting the mutational impact on the protein comprises:
selectively mutating the protein using single amino acid substitutions;
repeating steps (b)-(e) on the selectively mutated protein;
comparing the characterization of the selectively mutated protein and the protein from step 1.

17. The system of claim 10, wherein the system is used to investigate antibiotic resistance.

18. The system of claim 10, wherein the system is further configured to generate a report comprising the characterization of the at least one residue and the protein.

19. A non-transitory computer-readable medium having stored thereon instructions that, when executed by a processor to:
a) access simulated protein structure data, wherein the simulated protein structure data indicate a structure of the protein;
b) cluster the simulated protein structure data to produce a plurality of clustered structures and determine an equilibrium of each clustered structure in the plurality of clustered structures;
c) calculate time-dependent responses of each clustered structure in the plurality of clustered structures;
d) identify at least one residue of interest based on the time-dependent responses of each clustered structure in the plurality of clustered structures; and
e) characterize the at least one residue of interest and the protein.

20. The non-transitory computer-readable medium of claim 19, wherein accessing the simulated protein structure data comprises simulating the structure of the protein with the computer system using isobaric canonical ensemble MD simulations.

21. The non-transitory computer-readable medium of claim 19, wherein calculating the time-dependent responses of each clustered structure in the plurality of clustered structures comprises:
extracting cross velocity-positional covariance matrices corresponding to each clustered structure in the plurality of clustered structures; and
applying perturbative forces to the cross velocity-positional covariance matrices corresponding to each structure in the plurality of clustered structures to calculate the time-dependent responses.

22. The non-transitory computer-readable medium of claim 19, wherein identifying the at least one residue of interest comprises using frequency domain analysis.

23. The non-transitory computer-readable medium of claim 19, wherein identifying and characterizing the at least one residue of interest comprises identifying the at least one residue as allosteric or non-allosteric.

24. The non-transitory computer-readable medium of claim 19, wherein the non-transitory computer-readable medium is used to predict a mutational impact on the protein.

25. The non-transitory computer-readable medium of claim 19, wherein predicting the mutational impact on the protein comprises:
selectively mutating the protein using single amino acid substitutions;
repeating steps (b)-(e) on the selectively mutated protein;
comparing the characterization of the selectively mutated protein and the protein from step (a).

26. The non-transitory computer-readable medium of claim 19, wherein the non-transitory computer-readable medium is used to investigate antibiotic resistance.

27. The non-transitory computer-readable medium of claim 19, wherein the non-transitory computer-readable medium is further configured to generate a report comprising the characterization of the at least one residue and the protein.
